(19) United States
(12) Patent Application Publication
(10) Pub. No.: US 2011/0092906 A1
(43) Pub. Date: Apr. 21, 2011

(54) DEVICE WITH AT LEAST ONE CHAMBER FOR RECEIVING A MEDICAMENT OR A SAMPLE VOLUME

(75) Inventors: Frank Böttger, Ravensburg (DE); Benjamin Böbst, Mittelbiberach (DE)

(73) Assignee: ARZENEIMITTEL GMBH APOTHEKER VETTER & CO. RAVENSBURG, Ravensburg (DE)

(21) Appl. No.: 12/999,593

(22) PCT Filed: Jun. 18, 2009
(86) PCT No.: PCT/EP09/04388

§ 371 Date: Dec. 16, 2010

(30) Foreign Application Priority Data
Jun. 19, 2008 (DE) 10 2008 030 270.8

(51) Int. Cl. A61M 5/20

(52) U.S. Cl. 604/143

(57) ABSTRACT

A device has at least one chamber for accommodating a medicament or a sample volume, and has a plunger element which is displaceable in the device. The device includes a syringe or carpule, a multi- or dual-chamber system, an autoinjector, or a pen. Pressure forces resulting from a chemical reaction may be introduced into the plunger element, thereby causing displacement of the plunger element.

The invention relates to a device having at least one chamber for accommodating a medicament or a sample volume according to the preamble of Claim 1.

Devices of this type are known. A pneumatic injector is disclosed in WO 03/039634 A1, having a chamber for accommodating a medicament. A plunger element which is displaceable in the injector is also provided. When the injector is activated, displacement of the plunger element initially allows a needle to penetrate the skin of a patient. The subsequent displacement of the plunger element causes the medicament present in the chamber to be injected into the body of the patient. In this device, the displacement of the plunger element is brought about by connecting a previously closed reservoir, filled with pressurized carbon dioxide, to a chamber in which the plunger element is situated, so that the compressed carbon dioxide is able to exert pressure on the plunger element.

One disadvantage of such a mechanism is that the pressure necessary for a sufficiently rapid plunger motion must be stored over the entire storage period of the device. This places high demands on the seal-tightness of the reservoir for the carbon dioxide. If this reservoir has only slight leakage, the pressure may decrease over the storage period to such an extent that the device no longer functions. In addition, the pressure accumulator, i.e., the reservoir for the carbon dioxide, cannot be arbitrarily miniaturized, and thus imposes a lower limit for the size of the device. A further disadvantage is that the force required for advancing the plunger element is a function of various parameters. Typically, the aim is to keep the total duration of an injection fairly short so that the patient does not experience unnecessary inconvenience or discomfort. For introduction of total volumes, i.e., equal, complete total volumes of various medicaments into the body of a patient at the same time, for higher-viscosity medicaments a greater propulsion force is necessary for the plunger element, whereas less viscous medicaments require a lower propulsion force. The inner diameter of the injection needles used also plays a role: it is obvious that a greater propulsion force is necessary in order to convey the same quantity of a medicament, in the same time period, through a needle of lower diameter. To allow the device to be flexibly adapted to these various conditions, it would be necessary to adapt the pressure in the carbon dioxide reservoir to the particular conditions. However, for prefabricated pressurized carbon dioxide canisters produced in series this is possible only to a very limited extent, and at best, in the form of pressures ranges which are necessarily selected in a fairly inexact manner.

An autoinjector is disclosed in WO 2007/051331 A1 which likewise includes a chamber for accommodating a medicament, and a plunger element which is displaceable in the autoinjector. The plunger element is propelled by an elastic element, preferably a spring. One disadvantage of spring-operated devices of this type is that the chamber containing the medicament frequently is not completely emptied on account of an inadequate forward motion of the plunger element. The reason is that the elastic force introduced into the plunger element decreases over the path traversed by the plunger element. It is thus possible that when an injection is almost complete the elastic force is no longer sufficient to completely empty the chamber. As the result of tolerances in the springs that are used, this may result in particular in significant fluctuations in the administered dose. For spring-operated devices it is also disadvantageous that the elastic element introduces forces into the plunger element essentially in a relatively limited area. This is not a problem when the area of the introduction of force is situated approximately in the middle of the plunger element. However, if this is not the case, the elastic element applies a torque to the plunger element over the region of the introduction of force located further to the outside, as viewed in the radial direction, which may result in deformation or twisting of the plunger element. The injection device may thus be at least impaired in its function, and in the worst case, rendered completely unusable.

An infuser is known from US 2003/0168480 A1 which may be operated with the aid of gas propulsion. An infuser is a medical device by means of which a patient is to be injected with a preferably liquid medicament at a specified rate, i.e., a predefined volume per unit time. Examples of similar devices include a drip system and an electric syringe advance. It is not so much the overall total injected quantity or the total injected volume that is important, but, rather, very accurate maintenance of a specified injection quantity per unit time. The injection devices in question are typically exchanged before they are completely empty, so that the primary emphasis is not on complete emptying of the device. To be able to ensure a constant delivery rate of the medicament, the infuser necessarily requires a pressure regulator which relays the gas pressure, which is released as the result of a chemical reaction and adapted to the plunger element in such a way that the gas pressure is displaced at a desired, very precisely specified velocity.

In contrast, the devices addressed in the present patent application are intended to introduce a complete injection of a predefined volume of a medicament into a patient in a comparatively short period of time, or to be able to relatively quickly withdraw a sample volume which is defined as precisely as possible. However, a delivery or withdrawal rate which is as constant as possible is not of concern.

The object of the invention is to provide a device which does not have the referenced disadvantages.

The underlying object of the invention is achieved using a device having the features of Claim 1. The device, which includes a syringe or carpule, a multi- or dual-chamber system, an autoinjector, or a pen, is characterized in that pressure forces resulting from a chemical reaction may be introduced into the plunger element, causing displacement of the plunger element. In contrast to the devices referenced as prior art, the claimed device has a chamber for accommodating a medicament or a sample volume. This means that devices are also encompassed which are used for sampling. The plunger element is displaceable in a direction which is opposite the direction in which the plunger element is displaced when the device is used for administering a medicament. In this manner a sample may be introduced into the previously empty chamber, whereas for known devices, in which the chamber contains a medicament, the previously filled chamber is emptied during use. The mechanism by which the plunger element is displaceable in the device is essential for the device according to the invention. This displacement of the plunger element may basically take place in various directions. Thus, the device according to the invention may also be designed in such a way that a previously filled chamber is emptied during use, but may also be designed so that a previously empty chamber is filled during use. It is important that pressure forces resulting from a chemical reaction may be introduced into the plunger element. This means that the pressure forces which cause displacement of the plunger element are produced only at the moment of use, and are not present when the device is in the stored state. Decrease in the pressure which is present in the device during the storage period, and the resulting loss of functionality of the device, may thus be prevented. Thus, the requirements for seal-tightness of the device according to the invention are much less demanding than for devices which cause displacement of the plunger element as the result of pressure forces which are already present in the stored state.

A further advantage of the device according to the invention is that much less space must be provided for storing the substances which participate in the chemical reaction than for a customary carbon dioxide canister or some other pressure reservoir. The overall size of the device may therefore be smaller. In addition, the pressure forces generated by the chemical reaction may be precisely adapted to the desired conditions in a simple manner. This may be achieved, for example, by varying the chemical nature of the substances used, their overall quantity, or their mixing ratio. These parameters may be varied very easily by computer control on a modern production line, and thus allow practically continuous variation of the pressure forces which may be generated, so that these pressure forces may be individually adapted to the particular circumstances. Moving or pretensioned parts are largely eliminated, so that in this regard the device according to the invention is less susceptible to malfunction, and also smaller. The quantity of chemicals required is typically so small that the mechanism which causes displacement of the plunger element may be integrated very easily into the nonsterile regions of the device.

The pressure forces which may be introduced into the plunger element as a result of the chemical reaction and which cause displacement of the plunger element increase exponentially due to the kinetics of the chemical reaction. In contrast to an elastic element or a spring, which provides only a small elastic force toward the end of an injection, the pressure forces which may be introduced as a result of the chemical reaction increase toward the end of the injection. This ensures, with good reproducibility, that the entire contents of the chamber are always administered to the patient. On the other hand, during filling of the chamber for sampling it is ensured that the complete chamber volume is always filled.

A further advantage of the device according to the invention is that the pressure forces which may be introduced into the plunger element as a result of the chemical reaction are fully isotropic, i.e., act equally in all spatial directions or spatial angles. The forces which cause displacement of the plunger element are thus completely and uniformly distributed over the plunger element, so that no torque is applied thereto. Deformation or twisting of the plunger element, which may impair the function of the plunger element or even result in total failure thereof, is therefore prevented.

The chemical reaction proceeds independently of the geometry of the wall which encloses the reagents, so that the shape of the device may be adapted to the desired conditions in a very flexible manner. In contrast, the shape of known devices must always take into account geometries which are specified by the spring or a CO₂ canister.

Self-injecting devices such as autoinjectors, pens, self-injecting syringes or carpules, or multi- or dual-chamber systems have advantages for patients who have difficulty administering injections themselves due to fear of injections, or other impediments or disabling conditions. The systems in question are often designed in such a way that the patient does not see the needle present in the device, so that the typical anxiety reactions which are triggered by the mere sight of an injection needle may be avoided. Primarily autoinjectors or pens have this advantage. The term “autoinjector” generally refers to self-injecting systems, but is also frequently used for devices which are able to successively administer multiple doses. In comparison, the pen is able to administer only a single dose. Autoinjectors as well as pens may be designed as syringes or carpule syringes. The apparatus which causes displacement of the plunger element may also be separated from the rest of the device, so that the device has two separate parts. For example, one part of the device may include the chamber for accommodating a medicament or a sample volume, as well as the plunger element, while the other part includes the apparatus which causes displacement of the plunger element. This second part may be designed in such a way that the first part may be composed of a standard syringe or carpule, which may then be connected to the second part. The first part may also be a dual-chamber system. In this manner standard syringes, carpules, or dual-chamber systems may be connected to the second part of the device in such a way that the two parts together form the device according to the invention. The introduction of pressure forces into the plunger element as the result of a chemical reaction ensures that the injection is carried out quickly and completely.

A device is also preferred which is characterized in that in the course of the chemical reaction at least one gas is released which introduces pressure forces into the plunger element which cause displacement thereof. In principle, it is sufficient that during the chemical reaction a gas is released which due to its pressure is able to cause displacement of the plunger element. However, a chemical reaction may also be selected for which more than one gas is released, so that the resulting gases jointly introduce the pressure forces into the plunger element which cause displacement thereof.

Further advantageous embodiments result from the subclaims.

The invention is explained in greater detail below with reference to the drawings, which show the following:

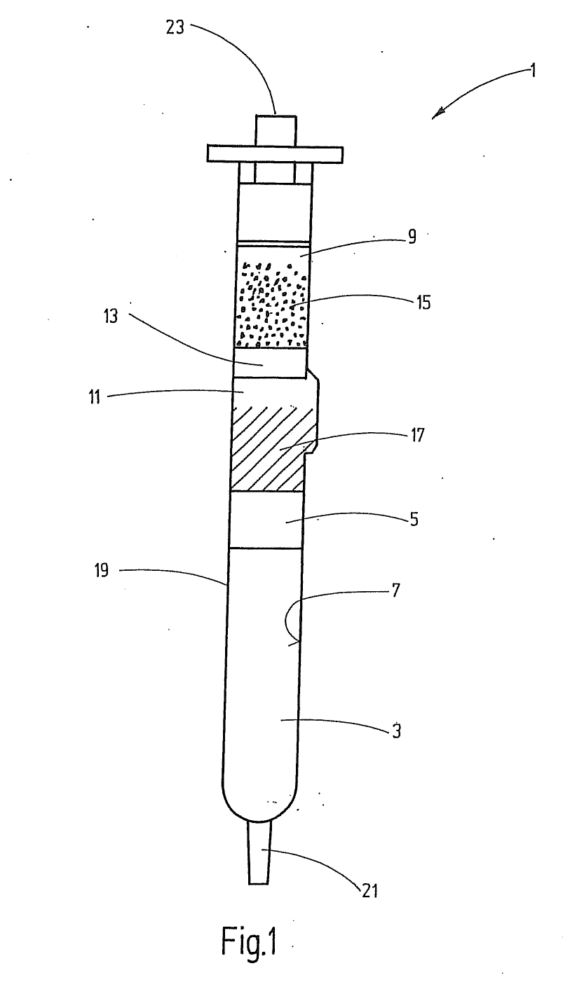

FIG. 1 shows a schematic view of a first exemplary embodiment of the device according to the invention, in its stored state;

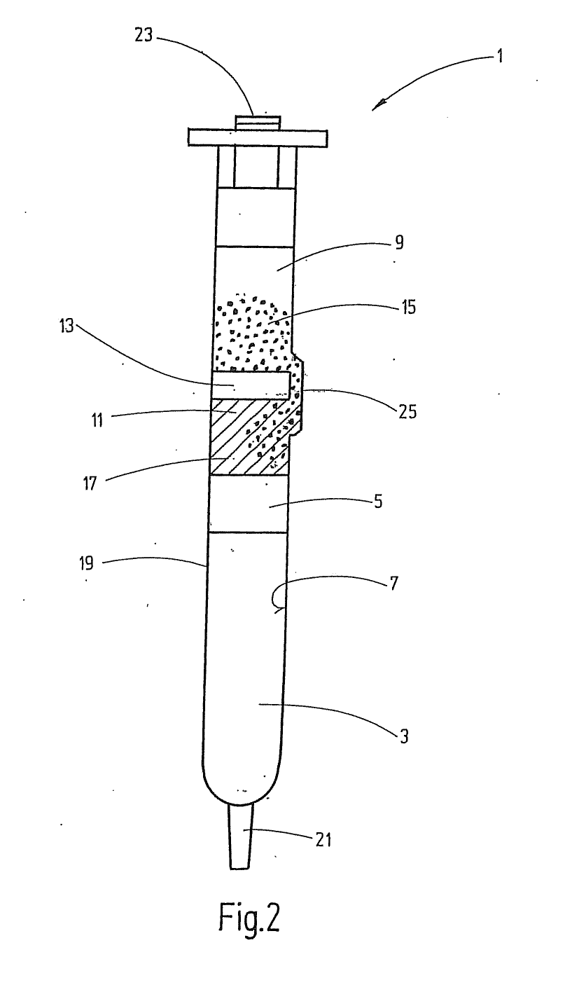

FIG. 2 shows the exemplary embodiment according to FIG. 1 during initialization of the chemical reaction;

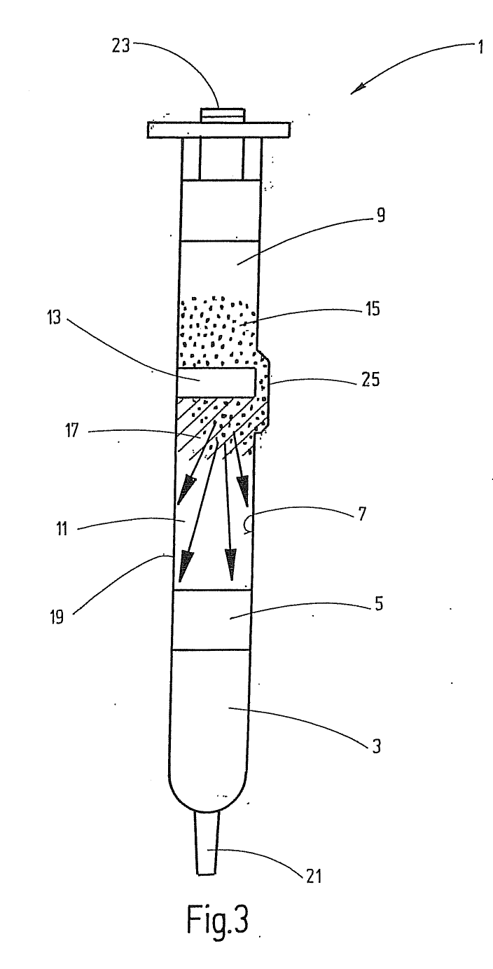

FIG. 3 shows the exemplary embodiment according to FIG. 1 during the course of the chemical reaction;

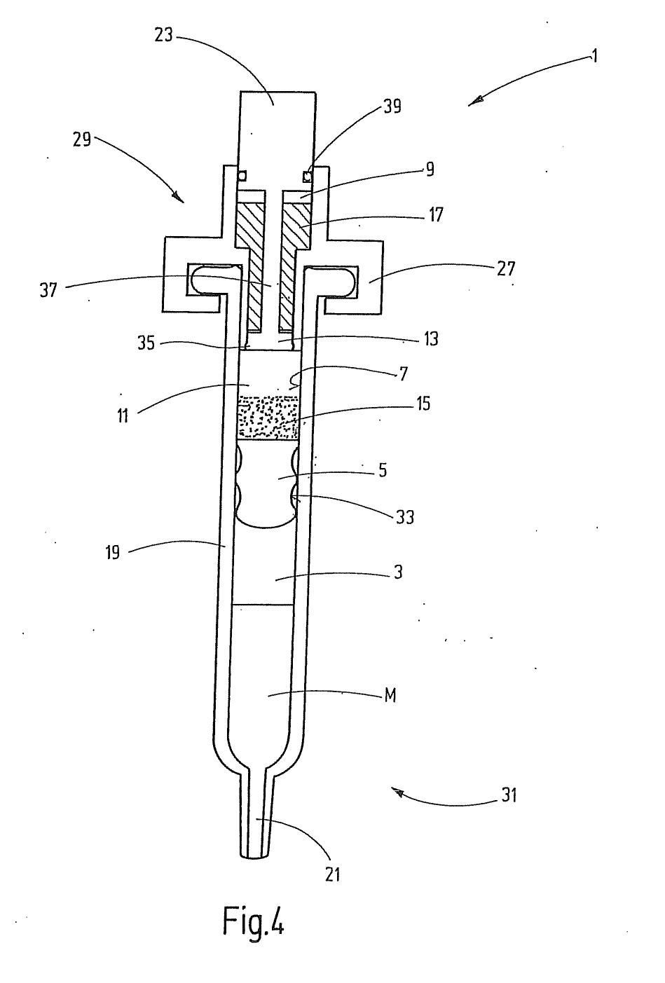

FIG. 4 shows a schematic illustration of another exemplary embodiment of the device according to the invention;

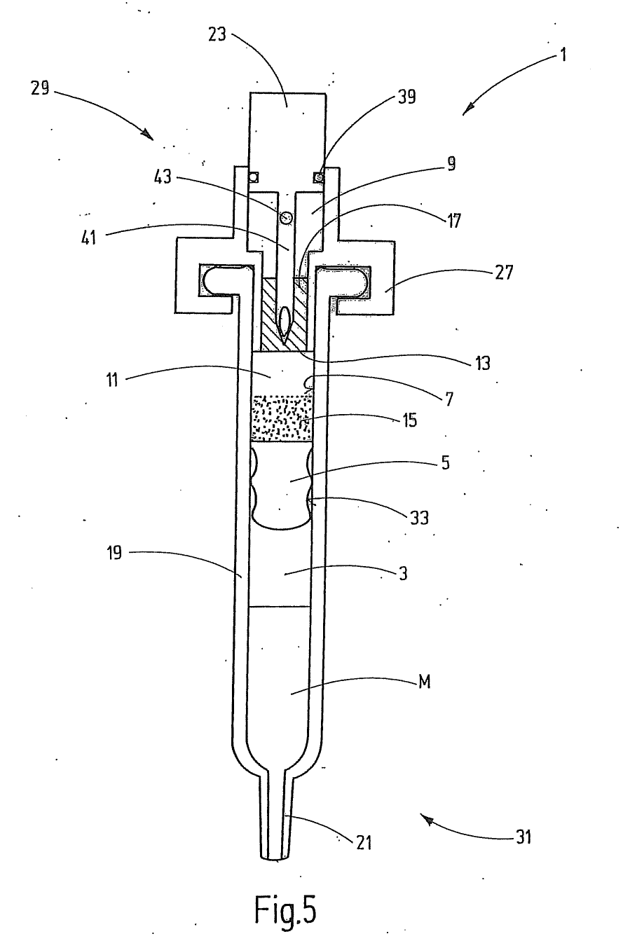

FIG. 5 shows a schematic illustration of a third exemplary embodiment of the device according to the invention;

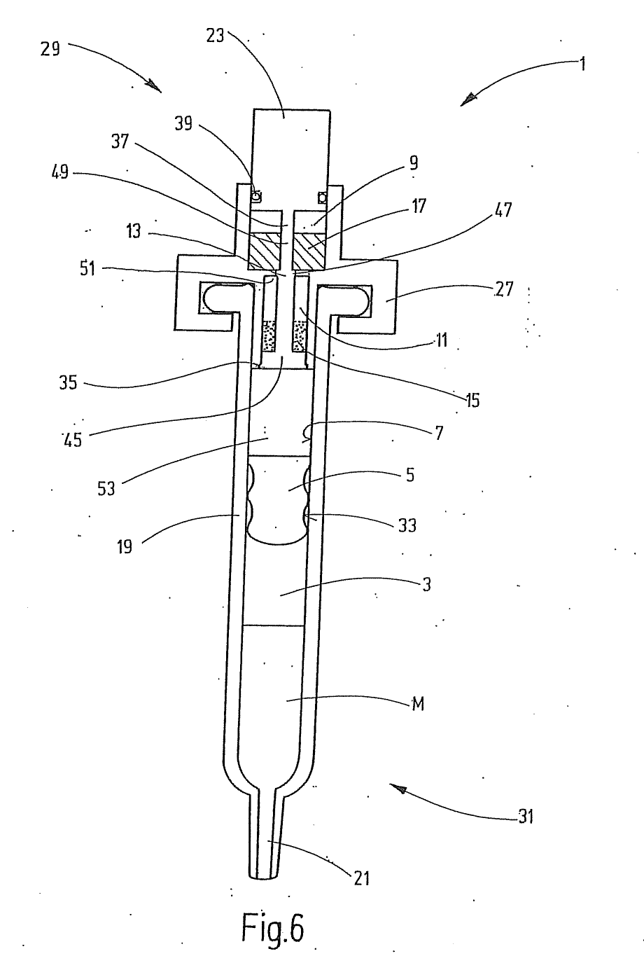

FIG. 6 shows a schematic illustration of a fourth exemplary embodiment of the device according to the invention;

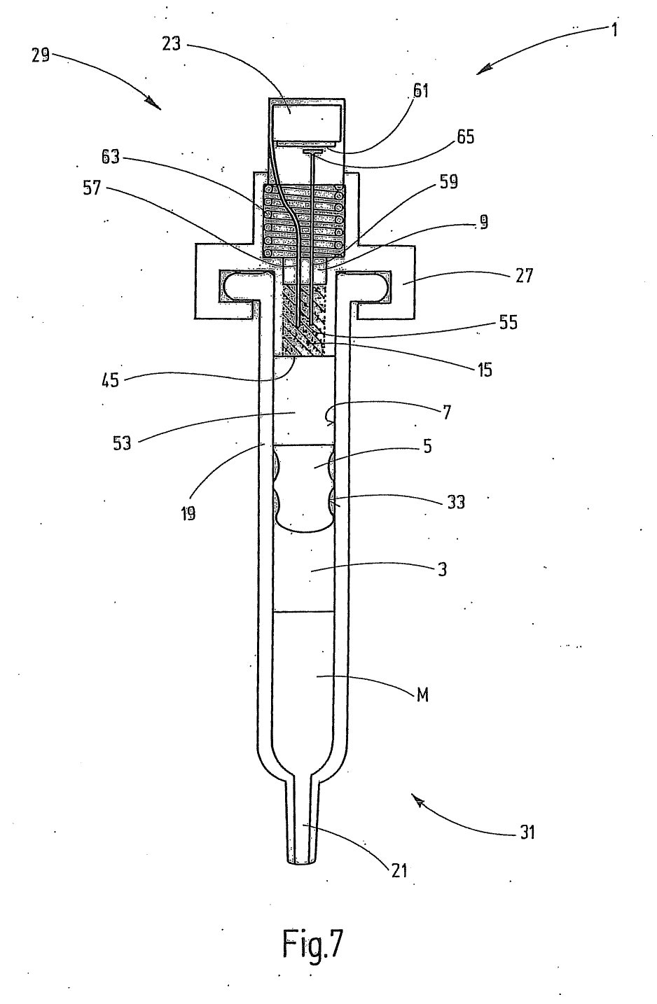

FIG. 7 shows a schematic illustration of a fifth exemplary embodiment of the device according to the invention;

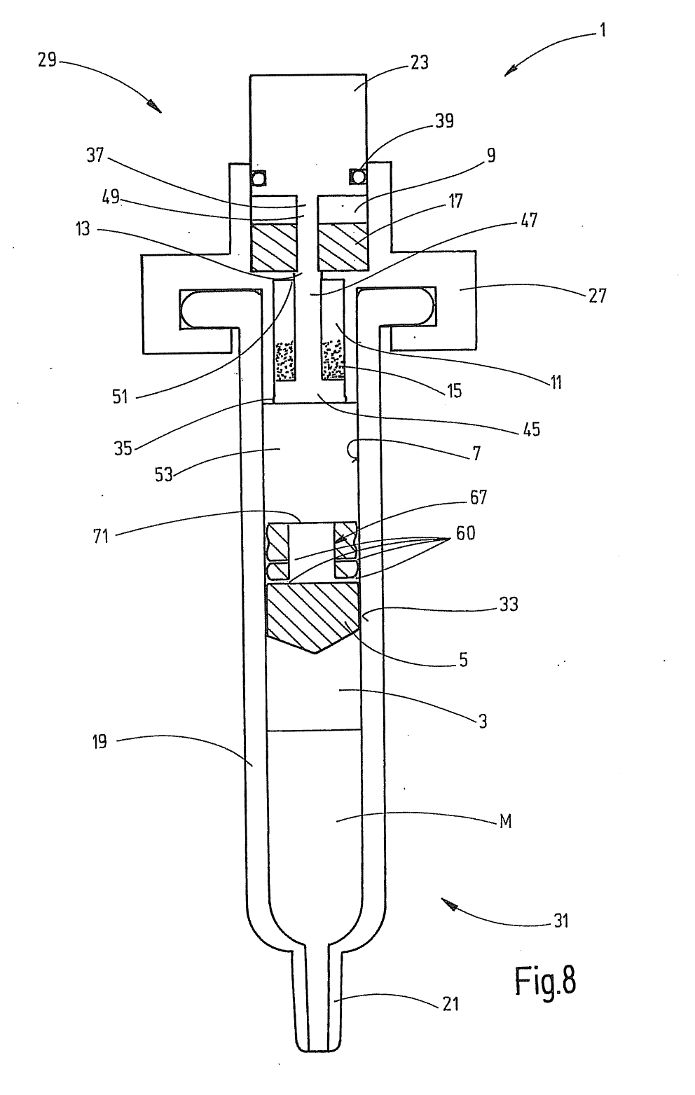

FIG. 8 shows a schematic illustration of the exemplary embodiment according to FIG. 6, wherein a special exemplary embodiment of a plunger element is provided; and

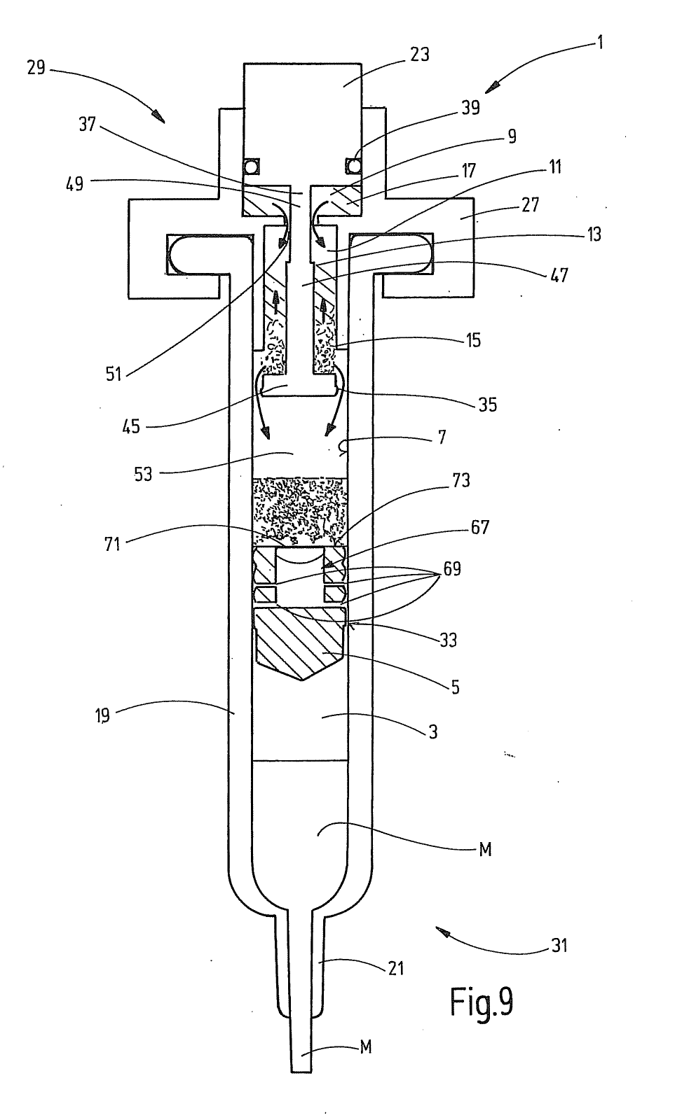

FIG. 9 shows the exemplary embodiment according to FIG. 8 during initialization of the chemical reaction.

FIG. 1 shows a schematic view of a first exemplary embodiment of the device 1 in its stored state. The device 1 is illustrated here as a syringe. However, the device 1 may also be a carpule, a carpule syringe, a multi- or dual-chamber system, an autoinjector, or a pen.

In the exemplary embodiment illustrated, the device 1 has a chamber 3 which contains a medicament, not illustrated. The chamber 3 is thus filled in the stored state of the device 1, so that the chamber may be emptied when the device 1 is activated. For this purpose a plunger element 5 is provided which is displaceable within the device 1. The plunger element 5 may, for example, be an elastomer stopper which sealingly closes the chamber 3 at one side due to the fact that the elastomer stopper makes sealing contact, at least in places, with the inner lateral surface 7 of the chamber 3. It is generally preferred that the plunger element 5 makes sealing contact with the inner lateral surface 7 of the chamber 3, at least in the region of the end of the plunger element facing the chamber 3, so that the chamber 3 is sealed with respect to the regions of the device 1 which are oppositely situated on the side of the plunger element 5 facing away from the chamber.

Pressure forces resulting from a chemical reaction may be introduced into the plunger element 5, causing displacement thereof. For this purpose the device 1 includes at least one space which accommodates at least one reagent for the chemical reaction. In the exemplary embodiment illustrated here, the device 1 includes a first space 9 and a second space 11. The spaces 9 and 11 are separated from another by a separating element 13. In the present exemplary embodiment, the separating element 13 is designed as a stopper which is displaceable within the device 1. However, the separating element may also be designed as a sealing plug, as a penetrable septum, or as a tearable or rupturable membrane. It is important that in the stored state of the device 1 the two spaces 9, 11 are reliably and consistently separated from one another by means of a separating element 13, and that for activating the device 1 they may be connected to one another, for example by displacing, penetrating, tearing, or breaking the separating element 13.

The first space 9 contains at least one reagent 15. The at least one reagent 15 may be present in liquid or solid form, and may be pulverized, for example. Of course, multiple reagents 15 may be present together in the first space 9, but it must be ensured that they do not react with one another 1, at least in the state in which they are present in the first space 9, during the storage period of the device. The second space 11 contains at least one substance 17, which may be at least one further reagent, or also a solvent, a solvent mixture, a solution, or at least one catalyst. It is also possible for the first space 9 to contain the at least one substance 17, while the second space 11 contains the at least one reagent 15. In the exemplary embodiment illustrated here, which includes two spaces 9, 11, it is important that a chemical reaction does not take place until the at least one reagent 15 is brought into contact with the at least one substance 17.

Other exemplary embodiments are also possible in which, for example, only one space 9 is provided which contains at least one reagent 15. The at least one reagent 15 may be a substance mixture whose substituents do not react with one another until an energy barrier is overcome. The chemical reaction may then be initiated, for example by thermal, photochemical, or electrochemical means, and/or by the action of a mechanical force, i.e., by introducing kinetic energy into the substance mixture. However, the at least one reagent 15 may also be a pure substance which may be decomposed by overcoming an energy barrier, wherein at least one gas may be evolved which introduces pressure forces into the plunger element 5.

In the present exemplary embodiment, the reagent 15 may be a pure substance which, for example, is able to react with another substance 17 with evolution of a gas. Due to the higher reaction rates, it is preferred that the at least one reagent 15 or the at least one substance 17 is present in the liquid phase. The at least one further reactant, which is situated in a separate space, may then be present as a solid, for example pressed into a pellet, or in powdered form. It is also possible for all of the substances participating in the reaction to be present in the liquid phase or in solution. In principle, all participants in the reaction may be present in the solid phase, although in some cases this may result in a retarded reaction rate.

The at least one reagent 15 may be a carbonate, for example sodium hydrogen carbonate. In this case it is preferred that the substance 17 is an acid, preferably an organic acid or mineral acid. The substance 17 may contain hydrochloric acid, for example, or may also contain a citric acid solution. In the latter case, mixture of the at least one reagent 15 with the at least one substance 17 would cause a neutralization reaction in which carbon dioxide is released.

It is generally preferred that the released gas is an inert and/or nontoxic gas. For example, carbon dioxide, nitrogen, oxygen, hydrogen, or methane may be formed.

If the at least one reagent 15 contains a mixture of reagents, the at least one substance 17 may, for example, include a solvent in which the reagents 15 are soluble. It is then possible that the reagents 15 do not react with one another when they are present in intermixed form in the solid phase, but react with evolution of gas when they are dissolved in a solvent 17. Of course, the at least one substance 17 may also contain a solution in which further reagents are dissolved which react with the at least one reagent 15, with evolution of gas. At least one catalyst may also be provided in at least one of the spaces 9, 11, which is able to lower a energy barrier for a reaction between the reagents or substances present in the separate spaces, to the extent that the reaction may be initiated when the reagents and substances are intermixed. Such a catalyst may be a metal, a metallic compound, or a biocatalyst, for example an enzyme.

As a whole, it must be ensured that in a device 1 according to the invention various reagents 15 may be present together in a space 9. A single reagent 15 may also be present in a space 9. A single space 9 may be provided, or further spaces 9, 11 having further reagents 15 and/or substances 17 may also be provided. Various reagents 15 or substances 17 which are at least partially separated from one another may be present in at least two spaces 9, 11. At least one solvent and/or at least one catalyst may also be provided. This catalyst may be present in at least one space 9, 11, but may also be provided in a separate space. The chemical reaction may be initiated by mixing the reagents 15 or substances 17 together, and/or by mixing the reagents 15 or substances 17 with at least one solvent and/or at least one catalyst. The reaction may also be initiated by overcoming an energy barrier. The reaction may be initiated by thermal, photochemical, or electrochemical means, and/or by the action of a mechanical force, i.e., by introducing kinetic energy into the reaction system.

It is apparent from FIG. 1 that the second space 11 is delimited by a base body 19 of the device and the plunger element 5. This is advantageous, since in the case that the reaction proceeds at least substantially in the second space 11, the released gas is able to directly introduce pressure forces into the plunger element 5 and thus displace same.

In this regard, it is particularly apparent that a pressure regulator which limits or regulates the pressure acting on the plunger element 5 may be dispensed with in the device 1. Instead, the gas released in the reaction is preferably introduced into the region of the plunger element 5 directly, i.e., at least without having previously passed through a pressure regulator, for example a control valve, so that the gas is able to introduce pressure forces into same. With regard to the devices 1 addressed here, such as syringes or carpules, multi- or dual-chamber systems, autoinjectors, or pens, for example, a pressure regulator may preferably be dispensed with, since it is not important, for example, that a medicament which is to be injected into a patient is injected at a precisely predefined injection rate. It is only important that a specified total volume is injected as quickly, and in particular as completely, as possible. However, the pressure forces which may be introduced into the plunger element 5 are preferably adapted to the specific conditions that are present, for example the viscosity of the medicament and the desired total duration of the injection. For this purpose, in one exemplary embodiment of the device a pressure regulator may be provided. However, it is preferred that the pressure forces are varied solely by the selection of the substances or reagents used, and/or by the selection of the quantities thereof.

Provided at one end of the chamber 3 is an attachment 21 for an apparatus which may be connected to the chamber 3 and which may act as a dispensing device for a medicament present in the chamber 3, or as a collection device for a sample flowing into the chamber 3. The apparatus may be a syringe needle, a canula, or a Braunula indwelling catheter, for example. Situated at the end of the device 1 facing away from the attachment 21 is an activating mechanism 23, by means of which the device 1 may be activated. In the present exemplary embodiment the activating mechanism 23 has a plug-shaped design and is displaceable within the device 1. In the stored state of the device 1 the activating mechanism 23 is located at a maximum distance from the attachment 21. To activate the device 1, the activating mechanism 23 may be moved within the device 1 in the direction of the attachment 21.

The mode of operation of the exemplary embodiment of the device according to the invention according to FIG. 1 is explained in greater detail below with reference to FIGS. 2 and 3.

FIG. 2 shows a schematic view of the exemplary embodiment according to FIG. 1 of the device 1. Identical and functionally equivalent elements are provided with the same reference numerals; therefore, in this regard reference is made to the preceding description. In this case the activating mechanism 23 is displaced by a user from its storage position, at a maximum distance from the attachment 21, to an activation position. The displacement causes the pressure in the first space 9 to increase, so that the separating element 13, in the present case designed as a displaceable stopper, is also displaced within the device 1 in the direction of the attachment 21. The base body 19 of the device 1 has a region of a larger inner diameter, encompassing only a small angular range in the circumferential direction, which forms a bypass 25. Along the longitudinal axis of the device 1 this bypass 25 has an extension which is larger than the extension of the separating element 13 in the same direction. In the stored state illustrated in FIG. 1, the separating element 13 is situated in the region of the bypass 25 in such a way that it sealingly closes access to the bypass 25 from the chamber 9. If the separating element 13 is then moved in the direction of the attachment 21, as shown in FIG. 2, the separating element reaches a position in which the bypass 25 is connected to both the first space 9 and the second space 11. Due to the fact that the bypass 25 covers only a small angular range in the circumferential direction, i.e., has a segmented design, the separating element 13 is reliably guided in this region as well by the inner lateral surface 7 of the device 1.

As a result of opening the bypass 25 which connects the first space 9 to the second space 11, the at least one reagent 15 may be transferred from the first space 9 to the second space 11, thus intermixing with the at least one substance 17. The chemical reaction may be initiated in this manner.

Instead of an external bypass 25 as described, it is also possible to connect the spaces 9, 11 by means of a bypass 25 which is situated in the interior of the device 1.

FIG. 3 schematically shows the exemplary embodiment of the device according to FIG. 1 during the course of the chemical reaction. Identical and functionally equivalent elements are provided with the same reference numerals; therefore, in this regard reference is made to the preceding description. As the result of intermixture of the at least one reagent 15 with the at least one substance 17 in the second space 11, a chemical reaction is initiated, during the course of which a preferably inert and/or nontoxic gas is released. The release of this gas causes the pressure in the second space 11 to increase, so that pressure forces are introduced into the plunger element 5, which is thus displaced within the device 1 in the direction of the apparatus 21. This also causes the pressure in the chamber 3 to increase, so that the medicament contained in the chamber 3 is dispensed through the attachment 21 and the apparatus connected thereto.

The speed at which the plunger element 5 is displaced within the device 1 depends on the kinetics of the chemical reaction. In addition, the force introduced into the plunger element 5 as a result of the pressure of the gas generated in the reaction is a function of the quantity of gas generated per unit time. Depending on the viscosity of the medicament contained in the chamber 3, the inner diameter of the apparatus 21, and the desired quantity of the medicament which is to be administered in a given time period corresponding to the total duration of the injection, the advancement of the plunger element 5 may be precisely adjusted to the particular needs. For this purpose, for example the type of chemical reaction or the participating reagents may be varied. In addition, for a given reaction the quantities of the substances used may be varied. In this regard, the total quantity of the substances as well as the various quantity ratios may be varied. Thus, the advancement of the plunger element 5 may be easily adapted to the individual requirements in a very precise manner. Furthermore, the propulsion mechanism may be scaled as desired, for example by the selection of the quantity of chemicals used, and may thus be used for very small devices as well as relatively large devices.

As a result of the kinetics of the chemical reaction which proceeds, the pneumatic force introduced into the plunger element 5 via the gas used as a propellant increases exponentially, so that, in contrast to known systems, complete emptying of the chamber 3 is always ensured. It is also apparent from the present exemplary embodiment that moving, mechanical, and pretensioned parts may be largely dispensed with. As a result, complicated, space-occupying components which are susceptible to malfunction are absent. The quantity of chemicals necessary for carrying out the reaction is generally so small that the propulsion mechanism may be adapted practically as desired to existing systems, or integrated into same. In particular, the propulsion system may be produced and installed completely independently from the aseptic technology, which is indispensable for the rest of the device. Namely, at no time does the propulsion mechanism come into any contact with the elements which themselves contact a patient.

It is apparent in the exemplary embodiment according to FIG. 1 that the spaces 9, 11 are integrally designed as part of the device 1. However, the subelement of the device 1 which causes the propulsion may be separated, at least partially, from the remainder of the device 1. In this case, at least one space for accommodating the reagents is provided separately from the device 1, and is connectable, preferably detachably connectable, to the device 1.

FIG. 4 schematically shows a second exemplary embodiment of the device, in which one space of the subelement of the device 1 which causes the propulsion is provided separately and is detachably connected to the remainder of the device 1, while a second space of the subelement which causes the propulsion is designed in one piece with the device 1. Identical and functionally equivalent elements are provided with the same reference numerals; therefore, in this regard reference is made to the preceding description. In this case, the subelement which causes the propulsion includes a retaining element 27 which is connectable, preferably detachably connectable, to the base body 19 of the device 1. In this manner, for example the part of the device 1 containing the medicament may be stored separately from the part which includes the retaining element 27. The part which includes the retaining element 27 may then be refilled and reused, for example, wherein shortly before the device 1 is used, for example said part is clipped or fastened in some other way to the part of the device 1 containing the medicament. The device 1 is thus divided into two parts: an upper part 29 and a lower part 31, in both cases from the viewpoint of the observer.

The first space 9 is situated in the upper part 29 of the device 1. In the present exemplary embodiment the first space contains at least one substance 17, which may be a solvent, solution, solvent mixture, or at least one reagent. The second space 11 is situated in the lower part 31 of the device 1, and contains at least one reagent 15. The space 11 is formed by the base body 19 of the device and the plunger element 5. In this case, an outer lateral surface 33 of the plunger element 5 has recesses and projections, the projections making sealing contact with the inner lateral surface 7 of the base body 19 of the device 1. In this exemplary embodiment as well, the at least one reagent 15 rests directly on the plunger element 5. In principle, a separating element may also be provided between the at least one reagent 15 and the plunger element 5, so that the at least one reagent 15 does not rest on the plunger element 5. In this case, the additional separating element is removed when the chemical reaction begins, for example by being ruptured by the pressure forces introduced into it, thus allowing the pressure forces to be introduced into the plunger element 5.

The spaces 9, 11 are separated from one another by a separating element 13. In the present case the separating element is designed as a sealing plug, the plug in its lower region (as viewed by the observer) having a sealing bead 35 which sealingly closes the first space 9. The plug-shaped separating element 13 also has a plunger rod 37 via which the separating element is connected to the activating mechanism 23. An annular groove 39 into which a sealing means, for example an O-ring, may be introduced is provided in the activating mechanism 23, thus allowing the space 9 to be sealed with respect to the activating mechanism 23.

The chemical reaction is initiated by displacing the activating mechanism 23 in the direction of the apparatus 21. In this case the plug-shaped separating element 13 is also displaced in the direction of the attachment 21, thus opening space 9 toward space 11. The at least one substance 17 contained in space 9 may then be intermixed with the at least one reagent 15 contained in space 11, thus initiating the chemical reaction. In the course of the chemical reaction the at least one gas is released, by means of which pressure forces are introduced into the plunger element 5, which is thus displaced within the device 1 in the direction of the attachment 21. This also causes the pressure in the chamber 3 to increase, so that the medicament M contained therein is dispensed through the attachment 21.

FIG. 5 shows a third exemplary embodiment of the device. Identical and functionally equivalent elements are provided with the same reference numerals; therefore, in this regard reference is made to the preceding description. In this exemplary embodiment as well, the device 1 has a first space 9 and a second space 11. The spaces 9, 11 are separated from one another by a separating element 13, which in the present case is designed as a penetrable septum. A hollow needle 41 which is connected to the activating mechanism 23 is situated in the upper space 9. In its upper region the hollow needle 41 has a borehole 43 via which the interior of the hollow needle 41 is connected to the space 9 surrounding the hollow needle 41.

The mode of operation of the present exemplary embodiment is as follows: When the activating mechanism 23 is displaced within the device 1 in the direction of the attachment 21, the hollow needle 41 perforates the septum 13, and thus penetrates from the upper space 9 into the lower space 11. The septum makes sealing contact with the circumferential face of the hollow needle 41, resulting in a connection between the spaces 9, 11 solely via the interior of the hollow needle 41. When the hollow needle 41 is displaced further in the direction of the attachment 21 by means of the activating mechanism 23, at a certain point the borehole 43 comes into contact with the at least one substance 17 present in the first space 9. This substance is able to pass through the borehole 43 and into the interior of the hollow needle 41, and via this path reaches the second space 11, where it comes into contact with the at least one reagent 15. In this manner the chemical reaction which results in the release of at least one gas may be initiated, thus introducing pressure forces into the plunger element 5.

The separating element 13 may also be designed as a tearable or rupturable membrane. In this case, instead of the hollow needle 41 a solid needle may be provided which causes tearing of the tearable membrane when the needle is displaced in the direction of the attachment 21 by means of the activating mechanism 23. If a rupturable membrane is provided as the separating element 13, either a solid needle or a solid breaking element may be provided which does not have a sharp end, for example. The solid needle or the breaking element are likewise connected to the activating element 23, thus allowing a user to break the separating element 13 when he introduces a sufficiently large force into the separating element 13 by means of the activating mechanism 23, via the solid needle or the breaking element.

FIG. 6 shows a fourth exemplary embodiment of the device. Identical and functionally equivalent elements are provided with the same reference numerals; therefore, in this regard reference is made to the preceding description. In the exemplary embodiment illustrated here, all elements of the apparatus which cause the propulsion of the plunger element 5 are integrated into the upper part 29 of the device 1, as viewed by the observer. This upper part is connected to the lower part 31 as viewed by the observer; the two parts 29, 31 are preferably detachably connected to one another.

The present and preceding exemplary embodiments show that the propulsion mechanism for the plunger element 5 may be present in a form which is integrated with the remainder of the device 1 (FIG. 1), or completely separate therefrom (FIG. 6). However, a subelement of the propulsion mechanism may also be integrated into the lower part 31 of the device 1, while another part is integrated into the upper part 29 of the device 1 (FIG. 5). If the propulsion mechanism is completely separable from the remainder of the device 1, it may also be manufactured in a separate production facility. The production facility for the lower part 31 of the device 1 may then be maintained under aseptic conditions, whereas this is not necessary for the production facility for the upper part 29. In this manner the aseptic technology is completely separated from the technology which is not required to be aseptic. In addition, commercial marketing of the upper part 29 may be conducted independently from the lower part 31. Thus, standard syringes, carpules, multi- or dual-chamber systems, autoinjectors, or pens may be used as the lower part 31, while the upper part 29 may be supplied or purchased separately. A detachable connection of the two parts 29, 31 of the device 1 with complete integration of the propulsion mechanism into the upper part 29 also allows the upper part 29, and thus the propulsion mechanism integrated at that location, to optionally be used multiple times, whereas the lower part 31 is intended for a single use. After the device 1 is used, it is possible, for example, to separate the upper part 29 from the lower part 31 and to refill the chemicals consumed, optionally after cleaning. The upper part 29 may then be reused with a new lower part 31.

As previously stated, in the present exemplary embodiment all elements of the propulsion mechanism are integrated into the upper part 29. In particular, in this case the retaining element 27 forms a base body of the part 29. This part has a first chamber 9 which contains at least one substance 17. The upper part also has a second chamber 11 which contains at least one reagent 15. The two chambers 9, 11 are separated from one another by a separating element 13, in the present case the separating element 13 being part of a plunger rod 37 of a closure element 45. The closure element 45 is essentially plug-shaped, and includes the plunger rod 37, which has a region 47 of larger diameter and a region 49 of smaller diameter. The region 47 of larger diameter engages with a recess 51 in the retaining element 27 used as a base body, thus forming a separating element 13 which separates space 9 from space 11. At its end facing the apparatus 21, the closure element 45 has an annular bead 35 which seals off the space 11 with respect to a third space 53, the third space 53 being delimited on the one hand by the base body 19 of the device 1 and on the other hand by the plunger element 5. The region 49 having a smaller diameter of the closure element 45 is connected to the activating mechanism 23.

When the activating mechanism 23 is displaced within the device 1 in the direction of the attachment 21, the region 47 having the larger diameter of the closure element 45 is moved out of the recess 51. Beyond a certain position, only the region 49 of smaller diameter is situated within the recess 51. Since the outer diameter of the region 49 of smaller diameter is smaller than the inner diameter of the recess 51, spaces 9 and 11 are thus connected to one another, allowing the at least one substance 17 to pass into space 11 and intermix with the at least one reagent 15 at that location.

At the same time, during displacement of the activating mechanism 23 in the direction of the attachment 21 the end of the closure element 45 facing the attachment is also moved in the same direction. As a result, the lower end of the closure element 45 also opens up space 11, so that the latter is connected to space 53. Thus, the at least one substance 17 and the at least one reagent 15 also pass into space 53; i.e., the at least one gas released as a result of the reaction, which possibly has already started, passes into space 53. Pressure forces are thus introduced into the plunger element 5, causing the latter to be displaced in the direction of the attachment 21. This also increases the pressure in the chamber 3, so that a medicament M contained therein is dispensed through the attachment 21.

FIG. 7 shows a fifth exemplary embodiment of the device. Identical and functionally equivalent elements are provided with the same reference numerals; therefore, in this regard reference is made to the preceding description. In this exemplary embodiment as well, the propulsion mechanism is completely integrated into the upper part 29 of the device 1. In the present case the upper part 29 includes a single space 9 which contains at least one reagent 15. The space 9 is separated via a closure element 45 from a space 53 which is also defined by the base body 19 of the device 1 and the plunger element 5. The chemical reaction of the at least one reagent 15 is inhibited by a energy barrier, which in this case, for example, may be overcome by thermal means. For this purpose, a heating element 55 having two electrodes 57, 59 is provided in the space 9. The activating mechanism 23 has a power source 61. This power source 61 may be formed by a battery, for example, preferably a button cell. A rechargeable accumulator is also possible. Solar cells may preferably be integrated into the device 1 which ensure that the power source 61 always has its nominal voltage when there is sufficient incidence of light. Since in this exemplary embodiment as well it is preferred that the propulsion mechanism together with the upper part 29 may be separated from the lower part 31 of the device 1, the propulsion mechanism may be stored in the presence of light, while the lower part 31 containing the medicament M may be stored with the exclusion of light.

Electrode 57 is permanently connected to one terminal of the power source 61, while electrode 59 may be connected to the other terminal of the power source 61. In the stored state of the device 1 or of the upper part 29, electrode 59 is not connected to its associated terminal of the power source 61. A spring element 63 introduces a pretensioning force into the activating mechanism 23, so that in the stored state the terminal of the power source 61 associated with the electrode 59 is always situated at a distance from the contact 65 associated with the electrode 59. When the activating mechanism 23 is displaced in the direction of the attachment 21, the terminal of the power source 61 associated with the electrode 59 makes contact with the contact 65. In this manner the electric circuit through the heating element 55 is closed, and the heating element is then able to supply heating power to the at least one reagent. The activation barrier for the chemical reaction may thus be overcome, and the reaction is initiated. The at least one gas released as a result of the reaction generates a pressure in the space 9, which upon reaching a certain threshold pressure causes the closure element 45 to open up a connection between space 9 and space 53. This may occur, for example, by tearing or breaking of the closure element 45. However, it is also possible for the closure element 45 to detach from the upper part 29 and fall into space 53. It is important that a connection is established between space 9 and space 53 so that at least one gas which is released as a result of the reaction is able to pass into the latter space and thus introduce pressure forces into the plunger element 5.

It is also possible to activate the chemical reaction electrochemically, for example, instead of thermally. For this purpose no heating element 55 would be provided; instead, the electrodes 57 and 59 would project into the at least one reagent 15, and initially form an open circuit when the activating mechanism 23 is activated. As a result of the potential applied to the electrodes 57, 59, an electrochemical reaction may be initiated, so that the circuit is ultimately closed by diffusion or migration of charge carriers along a potential gradient in the at least one reagent 15. The electrochemical reactions at the electrodes 57, 59 then allow initiation of a reaction, as a result of which at least one gas is released. At least one gas may also optionally be released directly as a result of the electrochemical reactions.

FIG. 8 shows an exemplary embodiment of the device 1 according to FIG. 6, which, however, includes a special exemplary embodiment of a plunger element 5. Identical and functionally equivalent elements are provided with the same reference numerals; therefore, in this regard reference is made to the preceding description. To allow displacement of the plunger element 5 despite the friction forces acting between its outer lateral surface 33 and the inner lateral surface 7, in the exemplary embodiments according to FIGS. 1 through 7 it is provided that the inner lateral surface 7 is coated, at least in places, with a lubricant, for example silicone, silicone oil, or a silicone oil emulsion. Otherwise, the friction forces for the materials typically used for the plunger element 5, preferably elastomers, would be so high that displacement of the plunger element 5 would hardly be possible. Even increasing the pressure forces introduced therein might not remedy the situation, since the relatively elastic material of the plunger element 5 would deform, thus increasing the friction forces between the outer lateral surface 33 and the inner lateral surface 7 even more. This would result in blockage of the plunger element 5, so that a further increase in pressure forces would be opposed by likewise progressively increasing friction forces. The plunger element 5 would then become stuck and would not be displaceable at all.

In the exemplary embodiment of a plunger element 5 illustrated in FIG. 8, coating of the inner lateral surface 7 with a lubricant may be eliminated. Namely, the plunger element 5 has a receiving area 67, which in the present case is designed as a cavity or reservoir and contains a lubricant. Channels 69¹ extend from the receiving area 67 to the outer lateral surface 33. In the exemplary embodiment illustrated, four channels 69 are evident. For other exemplary embodiments not illustrated, more than four channels may be provided, but in particular fewer than four channels 69 may also be provided. It has been shown that preferably at least one channel 69 may be provided which establishes a fluid connection between the receiving area 67 and the outer lateral surface 33, thus allowing lubricant to flow at this location. ¹Translator's note: Channels are denoted by reference numeral 60 in FIG. 8.

The reservoir 67 is sealed with respect to the third space 53 by at least one membrane 71 which is liquid-tight, but which at the same time is elastic and/or designed to be permeable to gases.

Thus, in the exemplary embodiment illustrated a self-lubricating plunger element 5 is realized, the mode of operation of which is explained in greater detail in conjunction with FIG. 9.

FIG. 9 shows the exemplary embodiment according to FIG. 8 during initialization of the chemical reaction. Identical and functionally equivalent elements are provided with the same reference numerals; therefore, in this regard reference is made to the preceding description. The mode of operation of the exemplary embodiment of a device according to FIG. 6 has already been explained in conjunction with that figure. Therefore only a brief summary is provided: When the activating mechanism 23 is moved downward in the direction of the attachment 21, a connection is established between the first space 9 and the second space 11, so that the reagent 15 and the substance 17 are able to come into contact and react with one another. At the same time, a fluid connection is also established between the second space 11 and the third space 53, so that at least the reaction mixture and the at least one gas released as a result of the reaction are able to pass into the third space 53. Positive pressure is thus generated at that location, which causes the plunger element 5 to be moved downward in the direction of the attachment 21. The fluid connections between spaces 9, 11 and spaces 11, 53 are indicated here by arrows.

As stated, the plunger element 5 may have a membrane 71 which is preferably permeable to gases, and which seals the receiving area 67 with respect to the third space 53 in a liquid-tight manner. The at least one gas released during the reaction is then able to permeate the membrane 71, resulting in pressure equalization between the receiving area 67 and the third space 53. The lubricant present in the receiving area 67 is thus acted on by the pressure, which expels it through the channels 69, as the result of which it is then available in the region between the outer lateral surface 33 and the inner lateral surface 7, and forms a lubricating film on which the plunger element 5 is able to slide.

Instead of a gas-permeable membrane 71, a membrane 71 which is elastic but impermeable to gases and liquids may preferably be used. Due to the pressure forces present in the third space 53, the membrane protrudes into the receiving area 67 and thus acts on the lubricant present at that location with a pressure which in turn expels the lubricant through the channels 69, so that it is available for forming a lubricating film between the lateral surfaces 33 and 7.

At the same time, the pressure present in the space 53 exerts a force on a surface 73 which causes the plunger element 5 to move downward on the attachment 21. The pressure forces released as a result of the chemical reaction thus act in two ways: first, they expel the lubricant, present in the receiving area 67, through the channels 69 so that a lubricating film results between the inner lateral surface 7 and the outer lateral surface 33; second, they cause displacement of the plunger element 5, which is then able to slide on the resulting lubricating film.

The downward displacement of the plunger element 5 causes the medicament present in the chamber 3 to be expelled through the attachment 21, which is schematically indicated here.

The combination of the gas propulsion according to the invention with a self-lubricating plunger element 5 has proven to be particularly advantageous. Namely, the pressure forces which continuously increase during the reaction ensure at the same time continuous expulsion of the lubricant as well as complete displacement of the plunger element 5, until a position is reached in which the desired injection volume is reliably dispensed. At the same time, coating the inner lateral surface 7 with a lubricant prior to completion and filling of the device 1 may be eliminated. This not only saves a work step, but also may be advantageous from a medical standpoint. Namely, it has been shown that the lubricants typically used may take part in undesired interactions, in particular with new, sensitive medicaments produced using biotechnology. For example, silicone oil together with proteins or peptides may result in aggregate formation or deposition. These aggregates are also suspected of triggering a number of adverse immune reactions in patients. In contrast, the self-lubricating plunger element 5 is able to guarantee that, at least during storage of the prefilled device 1, no contact occurs between the lubricant and the medicament M. In addition, during an injection, i.e., during displacement of the plunger element 5, there is preferably no contact between the lubricant and the medicament M, due to the fact that between the region of the plunger element 5 in which lubricant is supplied to the outer lateral surface 33 and the chamber 3 a sealing device, for example a circumferentially extending radial projection of the outer lateral surface 33, is provided which prevents the lubricant from entering the chamber 3.

Numerous exemplary embodiments of a self-lubricating plunger element 5 may be used with each of the exemplary embodiments of a device 1 described in the present patent application.

One exemplary embodiment may include, for example, a plunger element 5 which has a sponge saturated with lubricant as a receiving area. A sponge saturated with lubricant may also preferably be provided in the receiving area 67. In another exemplary embodiment the plunger element 5 may also have an overall design which is porous, in particular which may be wrung out or squeezed, and is able to absorb lubricant into its pores. The pressure forces acting on the lubricant then result in compression of the plunger element 5, so that the lubricant may be supplied to the outer lateral surface 33. At the same time, of course, in this exemplary embodiment displacement the plunger element 5 is brought about.

Instead of a sponge, a so-called microballoon may be used for absorbing the lubricant. The term “microballoon” refers to a volume which contains lubricant and is enclosed by a tearable cover. The cover may be torn either by pressure forces or inserting a needle, wherein the insertion of the needle is preferably caused by pressure forces, thereby releasing the lubricant.

It is preferably also possible to provide in the region of the receiving area 67 or the channels 69 a blocking device which makes it impossible for lubricant to flow in the channels 69 when the plunger element 5 is not under pressure. The blocking device also makes it possible for lubricant to flow in the channels 69 when pressure forces act on the plunger element 5. As the blocking device, a displaceable needle may preferably be provided which does not pass into a penetrable region when no pressure forces act on the plunger element 5. The pressure forces released upon initialization of the chemical reaction then cause displacement of the needle, so that it passes into the penetrable region, thus allowing lubricant to be supplied from a receiving area 67 via the needle. In other exemplary embodiments, instead of a needle a predetermined breaking point, a tearable membrane, a breakable material, or a lip seal which is closed without load may be used. The term “closed without load” indicates that the lip seal is pretensioned in such a way that it blocks a fluid connection between the receiving area 67 and the outer lateral surface 33 when no pressure forces act on it. The pressure forces released after initialization of the chemical reaction must first overcome the pretension of the lip seal before they open up the corresponding fluid connection, whereupon lubricant is able to flow from the receiving area 67 to the outer lateral surface 33.

In another exemplary embodiment, at least one sponge saturated with lubricant may be provided along the circumference of the plunger element 5 in such a way that the sponge is compressed when the plunger element 5 is introduced into the base body 19 of the device 1, and/or when the plunger element 5 is displaced within the device 1, so that lubricant may be supplied to the outer lateral surface 33.

Another exemplary embodiment provides that microspheres are introduced into the outer lateral surface 33 of the plunger element 5. The term “microsphere” refers to small, essentially spherical volumes which contain lubricant and are surrounded by a cover. This cover preferably is composed of the same material as the plunger element 5, or of the material of the plunger element 5 at least in the region of the outer lateral surface 33. The cover of the microspheres is preferably designed to be so thin that it tears when the plunger element 5 is displaced, and therefore sliding friction forces act on the outer lateral surface 33 and, thus, also on the microspheres present at that location. The microspheres are particularly preferably vulcanized into the material of the plunger element 5, which preferably includes an elastomer.

In another exemplary embodiment, the pressure forces of the device 1, which may be varied practically as desired, and which act continuously via the injection and are continuously released, may also advantageously be used to propel a plunger element 5 which does not have a self-lubricating design, but for which coating of the inner lateral surface 7 with lubricant is eliminated. To still be displaceable, in this case the plunger element 5 has a smooth, nonpolar surface, which preferably may be produced by coating. For example, the outer lateral surface 33 of the plunger element 5 may be coated with PTFE. It is preferred to provide a film made of perfluorinated plastic, for example PTFE, at least in the regions of the outer lateral surface 33 which are in contact with the inner lateral surface 7. Of course, for other types of coatings of the outer lateral surface 33 it is sufficient to coat at least the regions of the outer lateral surface 33 which are in contact with the inner lateral surface 7. However, a plunger element 5 is also preferred which is made completely of perfluorinated plastic, preferably PTFE.

It is obvious that in the latter exemplary embodiments of a plunger element 5 described, greater friction forces act between the outer lateral surface 33 and the inner lateral surface 7 than when a lubricant is used which is either applied beforehand to the inner lateral surface 7, or provided by a self-lubricating plunger element 5 during the injection. However, in this specific case the gas propulsion of the device 1 is advantageous because the pressure forces may be adapted to the particular conditions practically as desired, so that a sufficient force may be easily developed which is able to displace a plunger element 5, also without use of a lubricant, in such a complete and rapid manner that complete, rapid injection of the medicament M is ensured.

The use of a self-lubricating plunger element 5 or a plunger element 5 with complete elimination of a lubricant has been described only with regard to the dispensing of a medicament M. However, it is obvious that the mentioned exemplary embodiments of a plunger element 5 may be easily used in conjunction with sampling, in which a specified volume of a substance is introduced into the chamber 3 of the device 1.

The described exemplary embodiments share the common feature that a single-chamber system is involved, in the sense that only one chamber 3 is provided in which a medicament M is present. However, the device according to the invention is not limited to such single-chamber systems. The described propulsion mechanism may also be connected to a dual-chamber system, in which the active substances and/or adjuvants are present in separate chambers, or in which the active substances and/or adjuvants are present in separate chambers and separated by a solvent. The chambers may preferably be connected to one another when the device 1 is activated, so that the substances contained therein may be mixed together before the mixture may be dispensed to a patient through an attachment 21 and suitable devices. Typically, this connection of the two chambers is also directly or indirectly achieved by displacing at least one stopper into which pressure forces may be introduced. It is obvious that these pressure forces may also be introduced as the result of a chemical reaction. One exemplary embodiment is particularly preferred in which a two-stage propulsion mechanism is provided for a dual-chamber system. The propulsion mechanism is designed in such a way that double triggering is possible. The first triggering releases pressure forces which result in intermixture of the contents of the two chambers of the dual-chamber system. A second triggering releases pressure forces which result in expulsion of the mixed contents of the interconnected chambers through the attachment 21.

The subject matter of the previously described exemplary embodiments involves only the dispensing of a medicament M contained in a chamber 3. However, it is obvious that the device 1 may be changed by a relatively simple modification of its design in such a way that a plunger element 5 may be displaced in the opposite direction from an attachment 21 as the result of pressure forces generated by a chemical reaction. A negative pressure is thus generated in a chamber 3, so that a sample volume may be introduced into the chamber 3 via the attachment 21 and suitable devices. In this manner the device 1 according to the invention may be used for sampling. In the medical field this is practical, for example, for rapid withdrawal of blood samples. Patients who have a great fear of syringes, or also children, could thus have a blood sample withdrawn using a device 1 according to the invention, for example using a finger cuff, whereby with an appropriate design of the device 1 the needle is not visible to the patient throughout the entire withdrawal process. However, the device 1 may also be used for sampling in the environmental or chemical industry sectors, as well as in the food industry. The fields of application are in no way limited, and numerous situations are conceivable in which a device 1 according to the invention may be used for rapid, reliable, and defined sampling. Namely, by the selection of the chemical reaction or the total quantity or mixing ratio of the chemicals involved, the sample volume to be withdrawn may be adjusted very precisely.

The chemical reaction may also be selected in such a way that the reaction may be initiated by radioactive radiation. This may be advantageous in particular in the military sector. Thus, a soldier may be equipped with the device 1 in such a way that he carries the injection-ready device 1 directly on the body. If a weapon is deployed using radioactive radiation, the device 1 may be started by the released radiation, without the soldier having to take any action. Thus, the soldier may be automatically injected with a substance, an iodine preparation, for example, when this is necessary in a combat situation.

Accordingly, it has been shown that the device is based on a simple principle which has great advantages over the known propulsion mechanisms of similar conventional devices. In particular, development of the known propulsion mechanisms in adaptation to specific conditions is very complicated and costly. Thus, for example, it is difficult to adapt the pressure of carbon dioxide canisters to the specific exemplary embodiment of an injector containing a medicament of a given viscosity and a canula of a given diameter in such a way that a specified dose of the medicament may be dispensed per unit time. In contrast, an elastic or spring element which is more easily adapted to these requirements has the disadvantage that the elastic force decreases toward the end of the activation on account of the extension of the spring, so that complete functioning of the device is not ensured. Both of the known mechanisms are characterized by numerous complicated mechanical components which make miniaturization difficult and also require a complex design which is susceptible to malfunction. Furthermore, the known mechanisms are not very durable during storage of the device. Thus, for example, the pressure in a carbon dioxide reservoir may decrease as the result of carbon dioxide escaping through leakage points. A highly pretensioned spring may become fatigued during storage, so that the original force provided is no longer available when the device is to be used.

It is apparent from the preceding description that the device according to the invention does not have these disadvantages. In contrast, it may be used in a very flexible manner, does not impose special demands on the size or geometry of the installation space accommodating the propulsion mechanism, is easily adaptable to the specific conditions of use, and is very durable over a fairly long storage period. In addition, the gas pressure generated by the reaction increases as the reaction period progresses, so that when activation of the device 1 is almost complete, sufficient force is available to enable the desired operation, i.e., injection or sampling, to be carried out to completion. Not least of all, very simple, common chemicals such as a citric acid solution and carbonate-containing baking powder are possible as reagents. Furthermore, the propulsion mechanism according to the invention may be completely separated from the aseptic technology which is necessary for manufacturing the remainder of the device 1.

1-15. (canceled)

16. A device comprising:
at least one chamber for accommodating one of a medicament and a sample volume;
a plunger element displaceable within the device, and
an upper part and a lower part, the upper part detachably connectable to the lower part, the upper part including a propulsion mechanism by which pressure forces resulting from a chemical reaction may be introduced into the plunger element, thereby causing displacement of the plunger element;
wherein the lower part is selected from a syringe, a carpule, a multi chamber system, a dual-chamber system, an autoinjector, and a standard pen.

17. The device according to claim 16, wherein the propulsion mechanism is operative to release at least one gas during the chemical reaction for introducing pressure forces into the plunger element.

18. The device according to claim 16, wherein the device includes at least one space for accommodating at least one reagent for the chemical reaction.

19. The device according to claim 18, further comprising various reagents in the at least one space.

20. The device according to claim 18, wherein the device includes at least two spaces at least partially separated from each other for accommodating at least one reagent for the chemical reaction.

21. The device according to claim 19, further comprising at least one solvent and/or at least one catalyst in the at least one space.

22. The device according to claim 20, wherein the reaction is initiated by mixing reagents together, and/or by mixing reagents with at least one solvent and/or at least one catalyst.

23. The device according to claim 16, further comprising an energy barrier which is overcome to initiate the reaction.

24. The device according to claim 23, wherein the reaction may be initiated by thermal, photochemical, or electrochemical means, by radioactive radiation, and/or by the action of a mechanical force.

25. The device according to claim 20, wherein the spaces for reagents are separated from one another by a sealing plug which acts as a separating element.

26. The device according to claim 20, wherein the spaces for reagents are separated from one another by a penetrable septum which acts as a separating element.

27. The device according to claim 20, wherein the spaces for reagents may be separated from one another by a tearable or rupturable membrane which acts as a separating element.

28. The device according to claim 20, wherein the spaces for reagents are separated from one another by a displaceable stopper which acts as a separating element.

29. The device according to claim 20, wherein the spaces for reagents may be connected to another by means of a bypass.

30. The device according to claim 16, wherein the plunger element is a self-lubricating plunger element or a plunger element having a smooth, nonpolar surface, at least in places.
